(19) United States
(12) Patent Application Publication
(10) Pub. No.: US 2022/0136042 A1
(43) Pub. Date: May 5, 2022

(54) IMPROVED NUCLEIC ACID TARGET ENRICHMENT AND RELATED METHODS

(71) Applicant: Roche Sequencing Solutions, Inc., Pleasanton, CA (US)

(72) Inventors: Rui Chen, Fremont, CA (US); Toumy Guettouche, Pleasanton, CA (US); Donald E. Sharon, Bristol, NH (US)

(21) Appl. No.: 17/431,488

(22) PCT Filed: Feb. 19, 2020
(86) PCT No.: PCT/EP2020/054354

§ 371 Date: Aug. 17, 2021

Related U.S. Application Data
(60) Provisional application No. 62/808,734, filed on Feb. 21, 2019.

(51) Int. Cl. C12Q 1/6832; C12Q 1/686; C12Q 1/6876

(52) U.S. Cl. CPC C12Q 1/6832; C12Q 2600/156; C12Q 1/6876; C12Q 1/686

(57) ABSTRACT

The invention is an improved method of target enrichment and target depletion where probe binding is facilitated by the FANCA protein. Improved workflows for next regeneration sequencing and related methods involving nucleic acid probe hybridization are improved by the use of FANCA protein.

### FIELD OF THE INVENTION

The invention relates to the field of nucleic acid sequencing and more specifically, to target enrichment for high throughput single molecule nucleic acid sequencing.

### BACKGROUND OF THE INVENTION

High throughput sequencing technologies continue to find new uses in research and clinic. Modern methods are able to sequence an entire genome of an organism at a progressively lower cost. Many sequencing applications focus on only a portion of the genome or on a subset of all nucleic acids present in a sample. Target enrichment methods capture and optionally, amplify the desired nucleic acids for sequencing. Existing target enrichment methods require the hybridization of large populations of diverse single-stranded DNA (ssDNA) probes in order to capture and enrich for sequences of interest. Typically, a DNA sample is contacted with synthetic tagged DNA probes and annealed duplexes are then affinity purified. This process can be inefficient, imprecise and time consuming. In highly diverse capture pools, strand annealing is characterized by slow kinetics, so that hybridization reactions often require long incubation periods yet result in the capture of only a fraction of the desired targets. Current methods also suffer from non-specific capture of off-target sequences, resulting in reduced representation of the sequences of interest. As such, methods for improving the efficiency and accuracy of DNA enrichment assays are highly desirable.

Studies of defects in the double-strand break repair (DBR) pathway in patients with Fanconi anemia (FA) have led to the identification of multiple FA complementation group proteins (FANC proteins). These proteins are utilized in the single-strand annealing break repair pathway in which the 5′ strand at the end of either side of a break is resected and complementary repetitive sequences on the single-stranded 3′ ends are then annealed, processed, and closed by ligase. Benitez et al. (2018) FANCA promotes DNA double-strand break repair by catalyzing single-strand annealing and strand exchange, Molecular Cell 71:621 Eight FANC family member proteins are involved in the core complex, including Fanconi Anemia complementation group A (FANCA) which is the most frequently mutated protein in FA patients. FANCA has recently been demonstrated to exhibit high-level single-strand DNA strand annealing (SA) and strand exchange (SE) activity in vitro. Additional proteins including RAD52 family proteins assist in forming the complex between FANCA and nucleic acid strands, see Van den Bosch et al. (2002) DNA double strand break repair by homologous recombination, Biol. Chem. 383:873.

### SUMMARY OF THE INVENTION

The invention teaches improved hybridization of nucleic acids by carrying out a hybridization reaction in the presence of Fanconi Anemia complementation group A (FANCA) protein. Any method which comprises a nucleic acid hybridization step may be enhanced by the improvement disclosed herein, including without limitation, target capture or target detection by probe hybridization, target copying or target amplification, target sequencing workflows and in vitro recombination methods such as the CRSPR method.

In some embodiments, the invention is a method for capturing target nucleic acid sequences comprising: forming a reaction mixture comprising a nucleic acid sample which may or may not comprise one or more target sequences, a plurality of oligonucleotide probes at least partially complementary to the one or more target sequences, and Fanconi Anemia complementation group A (FANCA) protein; incubating the reaction mixture under conditions wherein hybridization between the one or more target sequences and the plurality of probes is catalyzed by the FANCA protein to form a plurality of target-probe hybrids. The nucleic acid sample may comprise genomic DNA or RNA target sequences. At least one of said target sequences may comprise a single nucleotide polymorphism (SNV) or a genomic copy number variant (CNV).

In some embodiments, the plurality of probes comprises probes conjugated to a capture moiety such as biotin. In some embodiments, the probes are affixed to a substrate. In some embodiments, the substrate comprises a ligand for the capture moiety, such as avidin or streptavidin. In some embodiments, the substrate is a microparticle or a microarray slide. In some embodiments the hybridization occurs on the solid phase.

In some embodiments, the plurality of probes comprises an interrogation nucleotide.

In some embodiments, the reaction mixture further comprises RAD52 protein or FANCG protein.

In some embodiments, the method further comprises separating the target-probe hybrids from the reaction mixture and releasing the targets from the target-probe hybrids and detecting said released target nucleic acid sequences by sequencing or detecting a detectable label such as a fluorescent label.

In some embodiments, the invention is a composition for sequence specific nucleic acid capture comprising one or more oligonudeotide probes and the Fanconi Anemia complementation group A (FANCA) protein.

In some embodiments, the invention is a kit for capturing nucleic acid sequences comprising one or more oligonudeotide probes and the Fanconi Anemia complementation group A (FANCA) protein. The kit may further comprise RAD52 protein or FANCG protein.

In some embodiments, the capture probe further comprises a detection moiety, such as a fluorescent moiety.

In some embodiments, the invention is a method of copying target nucleic acid sequences comprising forming a reaction mixture comprising a nucleic acid sample which may or may not comprise one or more target sequences, at least one oligonucleotide primer at least partially complementary to one or more target sequences, and Fanconi Anemia complementation group A (FANCA) protein; incubating the reaction mixture under conditions wherein hybridization between one or more target sequences and at least one primer is catalyzed by the FANCA protein, extending at least one primer thereby copying the one or more target sequences.

In some embodiments, the invention is a method of amplifying target nucleic acid sequences comprising forming a reaction mixture comprising a nucleic acid sample which may or may not comprise one or more target sequences, at least one pair of forward and reverse oligonucleotide primers at least partially complementary to the one or more target sequences, and Fanconi Anemia complementation group A (FANCA) protein; incubating the reaction mixture under conditions wherein hybridization between one or more target sequences and at least one pair of forward and reverse oligonucleotide primers is catalyzed by the FANCA protein, extending at least one pair of forward and reverse oligonucleotide primers in a series of cycles of primer extension, denaturation, primer hybridization and primer extension thereby amplifying one or more target sequences.

In some embodiments, the invention is a method of selectively depleting nucleic acids from a sample, the method comprising: contacting the sample with one or more oligonucleotide probes at least partially complementary to nucleic acids to be depleted and Fanconi Anemia complementation group A (FANCA) protein; incubating the sample under the conditions wherein hybridization between the nucleic acids to be depleted and the oligonucleotide probes is catalyzed by the FANCA protein; removing the complexes formed in step b) from the sample. The sequence to be depleted may be repeated sequence selected from human LINE and SINE, ribosomal or mitochondrial RNA or DNA or globin gene or cDNA sequence.

### BRIEF DESCRIPTION OF THE DRAWINGS

FIG. 1 depicts a dimer of the FANCA protein exhibiting its strand annealing (SA) activity.

FIG. 2 depicts a dimer of the FANCA protein exhibiting its strand exchange (SE) activity.

FIG. 3 illustrates an exemplary sequencing workflow where one or more steps are improved by the addition of the FANCA protein.

### DETAILED DESCRIPTION OF THE INVENTION

### Definitions

The following definitions aid in understanding of this disclosure.

The term “sample” refers to any composition containing or presumed to contain target nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA). The sample may be derived from an animal (including human), plant and fungal species. The sample may also be an environmental sample potentially comprising bacterial, archaeal or viral targets.

A term “nucleic acid” refers to polymers of nucleotides (e.g., ribonucleotides and deoxyribonucleotides, both natural and non-natural) including DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanosine, cytosine, uracil and thymidine) as well as non-natural bases. Some examples of non-natural bases include those described in, e.g., Seela et al., (1999) Helv. Chim. Acta 82:1640. The non-natural bases may have a particular function, e.g., increasing the stability of the nucleic acid duplex, inhibiting nuclease digestion or blocking primer extension or strand polymerization.

The terms “polynucleotide” and “oligonucleotide” are used interchangeably. Polynucleotide is a single-stranded or a double-stranded nucleic acid. Oligonucleotide is a term sometimes used to describe a shorter polynucleotide. An oligonucleotide may be comprised of at least 6 nudeotides or about 15-30 nudeotides. Oligonucleotides are prepared by any suitable method known in the art, for example, by a method involving direct chemical synthesis as described in Narang et al. (1979) Meth. Enzymol. 68:90-99; Brown et al. (1979) Meth. Enzymol. 68:109-151; Beaucage et al. (1981) Tetrahedron Lett. 22:1859-1862; Matteucci et al. (1981) J. Am. Chem. Soc. 103:3185-3191.

The term “primer” refers to a single-stranded oligonucleotide which hybridizes with a sequence in the target nucleic acid and is capable of acting as a point of initiation of synthesis along a complementary strand of nucleic acid under conditions suitable for such synthesis. The primer may be partially or perfectly complementary to the target nucleic acid as long as it can form a stable hybrid with the target and be extended by a nucleic acid polymerase. The term “forward and reverse primers” refers to a pair of primers complementary and to opposite strands of the target nucleic acids at sites flanking the target sequence. Forward and reverse primers are capable of exponentially amplifying the target by polymerase chain reaction (PCR).

The term “probe” refers to a single-stranded oligonucleotide (or a double-stranded oligonucleotide which is denatured into signal strands prior to use) which hybridizes with a sequence in the target nucleic acid and is capable of forming a stable hybrid with the target. The probe may be partially or perfectly complementary to the target nucleic acid as long as it can form a stable hybrid with the target under the hybridization conditions.

As used herein, the terms “target sequence”, “target nucleic acid” or “target” refer to a portion of the nucleic acid sequence in the sample which is to be detected or analyzed. The term target includes all variants of the target sequence, e.g., one or more mutant variants and the wild type variant.

The term “sequencing” refers to any method of determining the sequence of nucleotides in the target nucleic acid.

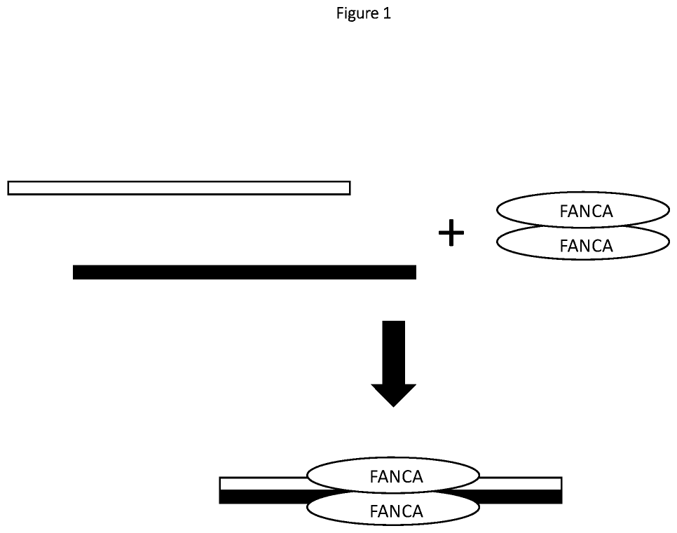

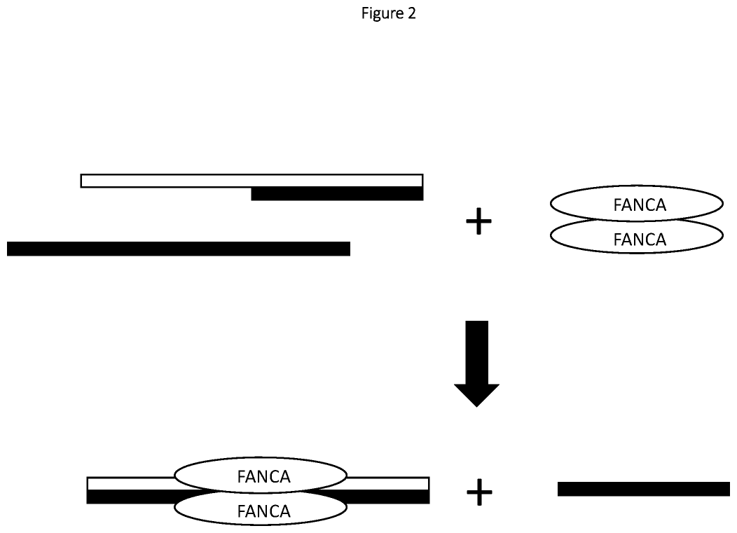

The present invention comprises a method of use of FANCA protein to improve the efficiency and accuracy of nucleic acid hybridization in various applications. FANCA facilitates annealing of a single strand to a nucleic acid duplex and strand exchange. This activity of FANCA is evidenced by the protein's ability to form homodimers and bind to single-stranded or partially single-stranded DNA oligomers. The single-stranded portions are then annealed to complementary single-stranded oligomers and strands are exchanged to form fully double stranded DNA species. (FIG. 1 and FIG. 2). FANCA's ability to cause strand annealing and strand exchange in vitro does not appear to be dependent on an energy source such as ATP or interactions with other FANC family members (although inclusion of purified FANCG protein has been shown to enhance the SA and SE activity of FANCA in in vitro assays).

In some embodiments, the reaction conditions favor the formation of a probe-target hybrid over other non-specific hybrids or partially complementary (partially matched) hybrids. FANCA acts as a catalyst to increase the rate of hybrid formation in the reaction.

In some embodiments, the invention is an improved method of target capture in current target enrichment workflows including target enrichment workflows which are part of the sequencing process, for example (without limitation) whole exome sequencing (WES). FANCA and optionally, an additional protein selected from FANCG and RAD52 (or both) are used as an additive during the hybridization step of target enrichment. In some embodiments, target enrichment is an in-solution sequence capture assay. In some embodiments, target enrichment utilizes capture ssDNA oligomers also referred to as bait oligonucleotides or oligos or capture probes. In some embodiments, the capture probes are tagged with a capture moiety (e.g., biotin) for later affinity capture. The present invention enables an increase in sample throughput by decreasing the time required for the hybridization reaction (typically performed over multiple hours or overnight). The present invention further improves the existing methods by allowing the target capture reaction to be performed at a lower temperature, for example, room temperature or even a lower temperature. Currently, to increase specificity, capture reactions are usually performed at temperatures above 45° C. In some embodiments, the instant method still comprises an initial target denaturation step at high temperature. However, the subsequent probe hybridization may take place at a lower temperature such as for example, room temperature or lower in the presence of the FANCA protein. In the method of the invention, during the capture probe hybridization step, FANCA protein catalyzes the annealing of complementary ssDNA species. Furthermore, during the capture probe hybridization step, FANCA protein catalyzes exchange of strands in partially matched or imperfectly matched hybrids for strands with higher levels of complementarity.

This approach significantly improves the state of the art target capture processes. The use of FANCA increases the efficiency and accuracy of hybrid capture reactions. The improvement in efficiency results in a reduction in total workflow time. The improvement in accuracy results in reduction of the sequencing depth required to achieve sufficient coverage of the target regions.

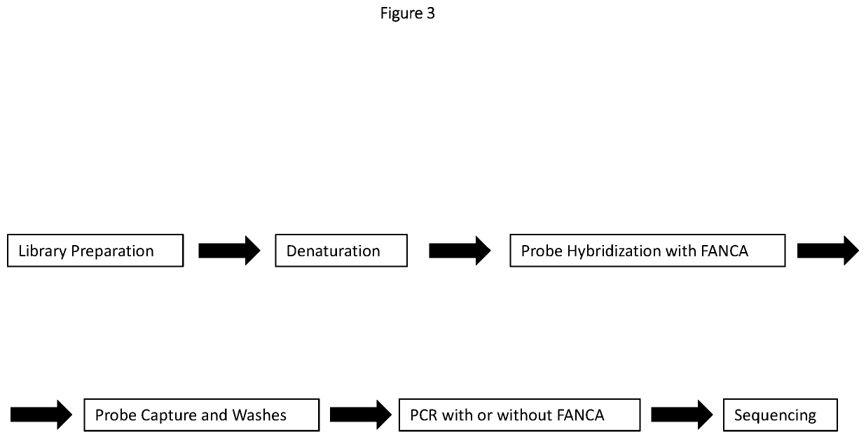

FIG. 1 depicts a dimer of the FANCA protein exhibiting its strand annealing (SA) activity wherein the protein dimer facilitates annealing of two complementary nucleic acid strands. FIG. 2 depicts a dimer of the FANCA protein exhibiting its strand exchange (SE) activity wherein the protein dimer facilitates replacing a partially complementary strand with a fully complementary strand. FIG. 3 illustrates an exemplary sequencing workflow where one or more steps are improved by the addition of the FANCA protein.

In some embodiments, the invention utilizes a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, and/or fecal samples). The sample may comprise whole blood or blood fractions where tumor cells may be present. In some embodiments, the sample, especially a liquid sample may comprise cell-free material such as cell-free DNA or RNA including cell-free tumor DNA or tumor RNA. The present invention is especially suitable for analyzing rare and low quantity targets. In some embodiments, the sample is a cell-free sample, e.g., cell-free blood-derived sample where cell-free tumor DNA or tumor RNA are present. In other embodiments, the sample is a cultured sample, e.g., a culture or culture supernatant containing or suspected to contain an infectious agent or nucleic acids derived from the infectious agent. In some embodiments, the infectious agent is a bacterium, a protozoan, a virus or a mycoplasma.

A target nucleic acid is the nucleic acid of interest that may be present in the sample. A plurality of different target nucleic acids may be present in the sample. The target may be a genomic sequence in the form of DNA or a transcribed sequence in the form of RNA, mRNA or cDNA. In some embodiments, the target nucleic acid is a gene or a gene fragment. In other embodiments, the target nucleic acid contains a genetic variant, e.g., a polymorphism, including a single nucleotide polymorphism or variant (SNP or SNV), or a genetic rearrangement resulting e.g., in a gene fusion. In some embodiments, the target nucleic acid comprises a biomarker. In other embodiments, the target nucleic acid is characteristic of a particular organism, e.g., aids in identification of the pathogenic organism or a characteristic of the pathogenic organism, e.g., drug sensitivity or drug resistance. In yet other embodiments, the target nucleic acid is characteristic of a human subject, e.g., the HLA or KIR sequence defining the subject's unique HLA or KIR genotype. In yet other embodiments, all the sequences in the sample are target nucleic acids e.g., in shotgun genomic sequencing.

In some embodiments, the nucleic acids in the sample comprise a library of nucleic acids formed for massively parallel sequencing. Such nucleic acids may comprise an insert sequence flanked by adaptors specific to a sequencing platform. In such embodiments, the probe nucleic acid is sufficiently complementary to the insert sequence to form a stable hybrid and enable capture, enrichment and depletion as described herein.

In some embodiments, the invention is a method of selectively depleting certain nucleic acids from the nucleic acids in the sample. The depletion enriches nucleic acids of interest for use in a downstream application such as amplification, sequencing and any further analysis. The depletion or removal of one or more nucleic acids from the sample uses probes and FANCA protein forming a complex between the probes and the nucleic acids to be depleted. The nucleic acid to be depleted may include overabundant sequence such as ribosomal RNA (rRNA) genes or transcripts, mitochondrial DNA (mtDNA) genes or transcripts, repetitive elements including LINE and SINE elements, as well as sequences or transcripts of highly expressed genes such as the globin gene.

The invention comprises a step of hybridizing a capture probe to the sequence to be enriched, captured or depleted from the sample. The probe is an oligonucleotide probe at least partially complementary to the extent of forming a stable hybrid with the target sequence. The binding or melting temperature (Tm) of the probe may be enhanced by incorporating one or more modified nucleotides into the probe in place of traditional nucleotides as follows:

| Standard NTP | Tₘ-modified substitute base |
|---|---|
| ATP | 8-aza-7-Br-7-deaza-2,6-diaminopurine |
| CTP | 5-propynyl-dC |
| GTP | 8-aza-7-Br-7-deaza-dG |
| TTP | 5-propynyl-dU |

Other nucleic acid modifications increasing the stability of the probe-target hybrid include backbone modifications such as locked nucleic acids (LNA).

In some embodiments, the invention is a method of using FANCA protein in hybrid capture assays wherein a sample is contacted with FANCA protein along with or prior to or after the addition of the capture probe. In some embodiments, the capture reaction takes place in solution wherein sample nucleic acid molecules (comprising the target nucleic molecules and non-target nucleic acid molecules), the capture probe and FANCA protein are present in solution. In some embodiments, the solution is enclosed in a microreactor such as a microwell, a microfluidic channel or reservoir or an oil encapsulated droplet which is a part of a water-in-oil emulsion.

In some embodiments, a target capture reaction contains biotinylated probes, sample nucleic acids and FANCA protein in a suitable hybridization buffer. The hybridization is conducted at lowered temperature (including ice), room temperature and higher temperatures, up to 45° C. The optimal temperature for a particular application may be determined experimentally. Optionally, one or both of RAD52 and FANCG protein is added to supplement the activity of the FANCA protein.

The hybridization reaction includes a hybridization buffer. The buffer can include 5 mM to 100 mM Tris-HCl (pH 6.5 to 8.5), 0 mM to 200 mM NaCl, 0 mM to 10 mM EDTA, 0 mM to 10 mM DTT, 0% to 20% glycerol, 0% to 40% DMSO, 0% to 2% Tween-20 (v/v), 0% to 10% Bovine Serum Albumin (w/v). Alternatively, the buffer can include 5 mM to 100 mM Na2HPO4 (pH 6.5 to 8.5), 5 mM to 100 mM K2HPO4 (pH 6.5 to 8.5), 0 mM to 10 mM KCl, 0 mM to 200 mM NaCl, 0 mM to 10 mM EDTA, 0 mM to 10 mM DTT, 0% to 20% glycerol, 0% to 40% DMSO, 0% to 2% Tween-20 (v/v), 0% to 10% Bovine Serum Albumin (w/v). Alternatively, the buffer can contain 10 mM to 3 M TMAC (pH 6.5 to 8.5), OM to 4 M Betaine, 0 mM to 200 mM MES, 0 mM to 10 mM EDTA, 0 mM to 10 mM DTT, 0% to 20% glycerol, 0% to 40% DMSO, 0% to 2% Tween-20 (v/v), 0% to 10% Bovine Serum Albumin (w/v).

The hybridization reaction also includes FANCA (1 nM to 2 mM) with or without FANCG (1 nM to 2 mM), and/or RAD52 (1 nM to 2 mM). The reaction further includes nucleic acids such as DNA or RNA from blood, tissue, including FFPE tissue, cell lines, cell-free circulating DNA, and/or synthetic or amplicon DNA to a concentration between 0.1 nM and 500 nM. The reaction further includes biotinylated or otherwise chemically modified nucleic acid probes (0.1 nM to 2 mM).

In other embodiments, one component among the sample nucleic acid molecules and the capture probe is attached to a solid support. In some embodiments, the solid support is a microparticle such as a magnetic or magnetizable particle including a glass or a polymer particle or bead such as superparamagnetic spherical polymer particles available as DYNABEADS™ magnetic beads (ThermoFisher, Scientific, Waltham, Mass.) or MAGPLEX® microspheres (Luminex, Austin, Tex.). In some embodiments, the solid support is a two-dimensional surface including without limitation, a slide or a microarray including an addressable microarray.

In some embodiments, the invention is a method of comparing genomes (comparative genomic hybridization, CGH) comprising genomic nucleic acids from one organism being fixed on a solid support and contacted with the genomic nucleic acids from another organism in a solution comprising FANCA protein.

In some embodiments, the invention is a method of detecting mutations including single nucleotide variations or polymorphisms (SNV or SNP) or copy number variations (CNV) comprising genomic nucleic acids from a reference genome being fixed on a solid support and contacted with the genomic nucleic acids from a test genome in a solution comprising FANCA protein. In some embodiments, the invention is a method of detecting mutations including single nucleotide variations or polymorphisms (SNV or SNP) or copy number variations (CNV) comprising genomic nucleic acids from a test genome being fixed on a solid support and contacted with the genomic nucleic acids from a reference genome in a solution comprising FANCA protein.

In some embodiments, the invention is a method of copying and optionally, amplifying target nucleic acids in a sample comprising annealing and extending of one or more target specific primers in a solution comprising the FANCA protein. In some embodiments, the method comprises copying one or both strands of the target nucleic acid by extending one or more target specific primers which hybridize to the target in the presence of the FANCA protein. In some embodiments, the method comprises amplifying one or both strands of the target nucleic acid by extending one or more target specific primers which hybridize to the target in the presence of the FANCA protein, wherein amplification is by a process of linear primer extension. In some embodiments, the method comprises amplifying one or both strands of the target nucleic acid by extending one or more target specific primers which hybridize to the target in the presence of the FANCA protein, wherein amplification is by a process of polymerase chain reaction (PCR) including all variations of PCR known in the art including without limitation, asymmetric PCR, long-PCR, allele-specific PCR, ligation mediated PCR, universal PCR, inverse PCR, hot-start PCR and the like.

An amplification reaction may include a buffer such as 1 mM to 300 mM Tris-HCl (pH=6 to 9), 1 mM to 100 mM MgCl₂, 0 mM to 1 M KCl, 0% to 5% Triton X-100 (v/v), 0% to 5% Tween-20 (v/v), template DNA (from blood, tissue, cell lines, FFPE, cell-free circulating DNA, viral DNA, cDNA, and synthetic or amplicon DNA) at 0.1 pg to bug (in a 25 uL reaction), 0.01 μM to 10 μM primers and a dNTP mixture: 0.01 mM to 20 mM. The reaction further comprises the appropriate amount and type of DNA polymerase depending on the application (e.g. long-range PCR, PCR for amplicon-based NGS, Hot-Start PCR, high fidelity PCR, etc.). The polymerase may be added as part of a master mix or after first denaturing the template DNA and primers. In some embodiments, a polymerase added with the master mix possesses hotstart capabilities, e.g., is inactive until the denaturation temperature is reached.

In some embodiments, an amplification reaction includes denaturation of the DNA sample at 65° C. to 100° C. for 1 second to 10 minutes, cooling to 0° C. to 45° C. and adding FANCA and/or FANCG and/or Rad52 proteins to the reaction followed by incubation for 5 seconds to 24 hours. Next, the primers may be extended for 5 seconds to 30 minutes at 0° C. to 80° C. In some embodiments, the reaction is further subjected to a standard thermocycler parameters, which will vary based on the length of the target sequences, type of polymerase employed, and desired yield.

In some embodiments, FANCA is used to improve methods of Primer Extension Target Enrichment (PETE). Multiple versions of PETE are described in U.S. application Ser. Nos. 14/910,237, 15/228,806, 15/648,146 and an International Application Ser. No. PCT/EP2018/085727. Briefly, the shared feature of the PETE methods is the first step of a one-round extension of a barcoded target-specific primer. In some embodiments, the invention is an improved PETE method wherein the binding of the target-specific primer to its target in a sample occurs in the presence of the FANCA protein. The primer binding catalyzed by FANCA is more specific and efficient compared to the state of the art.

In some embodiments, the invention further comprises a step of separating the captured nucleic acids from the sample comprising excess probes and non-target nucleic acids. In some embodiments, the capture step utilized the capture moiety conjugated to the probes and the ligand for the capture moiety. The capture moiety ay be selected from biotin and its equivalents (e.g., desthiobiotin) and the ligand may be selected from avidin and its equivalents (e.g., streptavidin). In some embodiments, the probe-target nucleic acid hybrids are captured and separated from the reaction mixture. Glass beads or polymer particles (DYNABEADS™ or MAGPLEX® microspheres) can be used to separate the bound target-probe hybrids.

In some embodiments, the target-probe hybrids formed in the presence of FANCA can be separated from non-target nucleic acids and excess probe by exonuclease digestion. For example, exonuclease VII and other single-strand specific exonuclease may be used.

In some embodiments, the invention is an improved method of decreasing the complexity of a genomic sample or a sample comprising a plurality of nucleic acid sequences with the use of blocking probes. Blocking probes are typically designed to bind to repetitive sequences (e.g., LINE and SINE). The blocking probes may also be designed to bind to a wild-type sequence that needs to be blocked in order to facilitate detection of a rare mutant sequence. In some embodiments, the invention is an improved method of reducing the complexity of a nucleic acid sample wherein the step of binding blocking probes to the nucleic acids in the sample is performed in the presence of FANCA protein. The blocking probe binding catalyzed by FANCA is more specific and efficient compared to the state of the art.

In some embodiments, the invention is a method of depleting one or more nucleic acids from the nucleic acids in a sample.

In some embodiments, the invention is an improved method of in vitro genetic recombination using the CRISPR-Cas system (Doudna J., Mali P., (2016). CRISPR-Cas: a laboratory manual. Cold Spring Harbor, N.Y.). The shared feature of the CRISPR methods is the initial step of hybridizing the guide RNA (gRNA) to the target sequence. In some embodiments, the invention is an improved in vitro CRISPR recombination method wherein the binding of the guide RNA to its target occurs in the presence of the FANCA protein. The gRNA binding catalyzed by FANCA is more specific and efficient compared to the state of the art.

In some embodiments, the invention further comprises a step of detecting the target nucleic acids captured by the capture probes. In some embodiments, the detection is by sequencing of the captured target nucleic acids. Sequencing can be performed by any method known in the art. Especially advantageous is the high-throughput single molecule sequencing capable of reading circular target nucleic acids. Examples of such technologies include the SOLiD platform (ThermoFisher Scientific, Foster City, Calif.), Heliscope fluorescence-based sequencing instrument (Helicos Biosciences, Cambridge, Mass.) Pacific BioSciences platform utilizing the SMRT (Pacific Biosciences, Menlo Park, Calif.) or a platform utilizing nanopore technology such as those manufactured by Oxford Nanopore Technologies (Oxford, UK) or Roche Sequencing Solutions (Roche Genia, Santa Clara, Calif.), via a reversible terminator Sequencing by Synthesis (SBS) (Illumina, San Diego, Calif.) and any other presently existing or future DNA sequencing technology that does or does not involve sequencing by synthesis.

In some embodiments, the invention is an improved sequencing workflow (FIG. 3). The typical sequencing workflow includes a library preparation step. This step comprises one or more instances of nucleic acid hybridization. During the target enrichment step, the one or more targets in the sample are hybridized to one or more capture probe sequences. In some embodiments, the invention is an improved sequencing workflow wherein the capture probe binding occurs in the presence of the FANCA protein. The probe binding catalyzed by FANCA is more specific and efficient compared to the state of the art and thus the target enrichment step and the entire sequencing workflow is improved.

In some embodiments, the sequencing workflow includes an amplification step with either target specific or universal primers. In this step, one or more primer pairs annealing to the target nucleic acids. In some embodiments, the invention is an improved sequencing workflow wherein the amplification primer annealing occurs in the presence of the FANCA protein. The primer annealing catalyzed by FANCA is more specific and efficient compared to the state of the art and thus the amplification step and the entire sequencing workflow is improved.

In some embodiments, the capture probe comprises a fluorescent moiety which can be detected with a suitable device. In some embodiments, an enzyme-based detection system is used and an enzyme substrate is conjugated to the detection probe. In some embodiments, the probe-target nucleic acid hybrids with detectable moieties are situated on a two-dimensional solid support where they can be detected.

In some embodiments, the invention is a composition or a reaction mixture for capturing target nucleic acids. The novel composition or a reaction mixture comprises one or more target nucleic acids, one or more probes at least partially complementary to the targets and the FANCA protein. Optionally, the composition or a reaction mixture further comprises one or more additional proteins known to facilitate strand hybridization or enhance FANCA activity. The additional proteins may be RAD52 and FANCG.

In some embodiments, the invention is a kit for capturing target nucleic acids. The novel kit comprises one or more probes at least partially complementary to the targets and the FANCA protein. Optionally, the kit further comprises additional proteins known to facilitate strand hybridization or enhance FANCA activity. The additional proteins may be RAD52 and FANCG. In some embodiments, the kit includes a set of custom probes for a particular set of target nucleic acids of interest to a customer. In some embodiments, the kit includes a set of probes for a particular application. For example, the kit may comprise one or more sets of probes for targets relevant to cancer diagnosis, monitoring and therapy selection. An example of such sets of probes are the probes in AVENIO ctDNA Analysis kits (Roche Sequencing Solutions, Inc., Pleasanton, Calif.). The kit may further comprise hybridization buffers, wash buffers and a set of instruction for performing hybridization in the presence of the FANCA protein according to the method disclosed herein.

In some embodiments, the invention is a composition or a reaction mixture for copying or amplifying target nucleic acids. The novel composition or a reaction mixture comprises one or more target nucleic acids, one or more primers or primer pairs capable of driving copying or amplification of the targets and the FANCA protein. Optionally, the composition or a reaction mixture further comprises one or more additional proteins known to facilitate strand hybridization or enhance FANCA activity. The additional proteins may be RAD52 and FANCG.

In some embodiments, the invention is a kit for copying or amplifying nucleic acids. The novel kit comprises one or more primers or primer pairs capable of driving copying or amplification of the targets and the FANCA protein. Optionally, the kit further comprises additional proteins known to facilitate strand hybridization or enhance FANCA activity. The additional proteins may be RAD52 and FANCG. In some embodiments, the kit includes a set of custom primers for a particular set of target nucleic acids of interest to a customer. In some embodiments, the kit includes a set of primers for a particular application. For example, the kit may comprise one or more sets of primer for targets relevant to testing for infectious diseases. An example of such sets of primers are the primers in COBAS® TaqScreen MPX Test kits (Roche Molecular Solutions, Inc., Pleasanton, Calif.). The kit may further comprise amplification buffers, dNTPS, a nucleic acid polymerase and a set of instruction for performing copying or amplification of the targets in the presence of the FANCA protein according to the method disclosed herein.

### EXAMPLES

### Example 1 (Prophetic) Improved Method of Target Capture Using FANCA Protein

In this example, a typical target capture reaction contains biotinylated probes, sample nucleic acids and FANCA protein in a suitable hybridization buffer.

The hybridization is conducted at lowered temperature (including ice), room temperature and higher temperatures, up to 45° C. Optionally, one or both of RAD52 and FANCG protein is added to supplement the activity of the FANCA protein.

The hybridization reaction includes a buffer such as: 5 mM to 100 mM Tris-HCl (pH 6.5 to 8.5), 0 mM to 200 mM NaCl, 0 mM to 10 mM EDTA, 0 mM to 10 mM DTT, 0% to 20% glycerol, 0% to 40% DMSO, 0% to 2% Tween-20 (v/v), 0% to 10% Bovine Serum Albumin (w/v). Alternatively, the buffer includes: 5 mM to 100 mM Na2HPO4 (pH 6.5 to 8.5), 5 mM to 100 mM K2HPO4 (pH 6.5 to 8.5), 0 mM to 10 mM KCl, 0 mM to 200 mM NaCl, 0 mM to 10 mM EDTA, 0 mM to 10 mM DTT, 0% to 20% glycerol, 0% to 40% DMSO, 0% to 2% Tween-20 (v/v), 0% to 10% Bovine Serum Albumin (w/v). Alternatively, the buffer includes: 10 mM to 3M TMAC (pH 6.5 to 8.5), 0M to 4M Betaine, 0 mM to 200 mM MES, 0 mM to 10 mM EDTA, 0 mM to 10 mM DTT, 0% to 20% glycerol, 0% to 40% DMSO, 0% to 2% Tween-20 (v/v), 0% to 10% Bovine Serum Albumin (w/v).

The hybridization reaction also includes FANCA (1 nM to 2 mM) with or without FANCG (1 nM to 2 mM), and/or RAD52 (1 nM to 2 mM). The reaction further includes nucleic acids such as DNA or RNA from blood, tissue, including FFPE tissue, cell lines, cell-free circulating DNA, and/or synthetic or amplicon DNA to a concentration between 0.1 nM and 500 nM. The reaction further includes biotinylated or otherwise chemically modified nucleic acid probes (0.1 nM to 2 mM).

Strand exchange/strand annealing reactions are conducted on ice or at temperatures between 0° C. to 50° C. Reactions times is between 1 minute and up to 24 hours.

Optionally, at each temperature, FANCA mutants that do not possess strand annealing or strand exchange activity (e.g., FANCA-F1263Δ) are used as negative controls. Duplexes containing target nucleic acids and capture probes are captured with a capture molecule such as streptavidin or any capture molecule specific for the probe modification, released and sequenced. Sequence is analyzed to determine coverage and representation of the target nucleic acids.

### Example 2 (Prophetic) Improving PCR Using FANCA Protein

In this example, a typical PCR reaction contains PCR primers, sample nucleic acids, thermostable polymerase and dNTPs in a suitable buffer with ions and any co-factors necessary for the polymerase action, and FANCA protein (FANCA (1 nM to 2 mM) with or without FANCG (1 nM to 2 mM), and/or RAD52 (1 nM to 2 mM). PCR is conducted under standard conditions using a suitable PCR temperature profile. The primer annealing step of the PCR temperature profile may be modified (i.e., shortened, eliminated or conducted at a different temperature) to account for improved annealing in the presence of the FANCA protein. The amplified products are analyzed by a suitable detection method.

In this example, the buffer is 1 mM to 300 mM Tris-HCl (pH=6 to 9), 1 mM to 100 mM MgCl₂, 0 mM to 1 M KCl, 0% to 5% Triton X-100 (v/v), 0% to 5% Tween-20 (v/v), template DNA (from blood, tissue, cell lines, FFPE, cell-free circulating DNA, viral DNA, cDNA, and synthetic or amplicon DNA) at 0.1 pg to 10 ug (in a 25 uL reaction), 0.01 μM to 10 μM primers and a dNTP mixture: 0.01 mM to 20 mM and a DNA polymerase added as part of a master mix.

The amplification reaction includes denaturation of the DNA sample at 65° C. to 100° C. for 1 second to 10 minutes, cooling to 0° C. to 45° C. and adding FANCA and/or FANCG and/or Rad52 proteins to the reaction followed by incubation for 5 seconds to 24 hours. Next, the primers may be extended for 5 seconds to 30 minutes at 0° C. to 80° C. In some embodiments, the reaction is further subjected to a standard thermocycler parameters, which will vary based on the length of the target sequences, type of polymerase employed, and desired yield.

### REFERENCES

Benitez, A., et al (2018). FANCA Promotes DNA Double-Strand Break Repair by Catalyzing Single-Strand Annealing and Strand Exchange. Molecular Cell, 71, 621-628.

Bhargava, R., Onyango, D., and Stark, J. (2016). Regulation of Single-Strand Annealing and its Role in Genome Maintenance. Trends in Genetics, Vol. 32, No. 9, 566-575.

Ceccaldi, R., Rondinelli, B., and D'Andrea, A. (2016) Repair Pathway Choices and Consequences at the Double-Strand Break. Trends in Cell Biology. Vol. 26, No. 1, 52-64.

Palovcak, A., et al (2018) Stitching up broken DNA ends by FANCA. Mol Cell Oncol. Vol. 5, No. 6.

1. A method for capturing nucleic acid sequences comprising:
(a) forming a reaction mixture comprising:
(i) a nucleic acid sample, which may or may not comprise one or more target sequences,
(ii) one or more oligonucleotide probes, wherein the one or more oligonucleotide probes is at least partially complementary to the one or more target sequences, and
(iii) one or more Fanconi Anemia complementation group A (FANCA) proteins, and
(b) incubating the reaction mixture under conditions wherein hybridization between the one or more target sequences and the one or more oligonucleotide probes is catalyzed by the one or more FANCA proteins to form a plurality of target-probe hybrids.

2. The method of claim 1, wherein the nucleic acid sample comprises genomic DNA.

3. The method of claim 1, wherein the nucleic acid sample comprises RNA target sequences.

4. The method of claim 1, wherein at least one of the one or more target sequences comprises a single nucleotide polymorphism (SNV).

5. The method of claim 1, wherein at least one of the one or more target sequences comprises a genomic copy number variant (CNV).

6. The method of claim 1, wherein the one or more oligonucleotide probes comprises probes conjugated to a capture moiety.

7. The method of claim 1, wherein one or more oligonucleotide probes are affixed to a substrate.

8. The method of claim 1, wherein the one or more oligonucleotide probes comprises an interrogation nucleotide.

9. The method of claim 1, wherein the reaction mixture further comprises one or more RAD52 protein or one or more FANCG protein.

10. A composition for sequence specific nucleic acid capture comprising: (a) one or more oligonucleotide probes, and (b) one or more Fanconi Anemia complementation group A (FANCA) proteins.

11. A kit for capturing nucleic acid sequences, wherein the kit comprises: (a) one or more oligonucleotide probes, and (b) one or more Fanconi Anemia complementation group A (FANCA) proteins.

12. The method of claim 1, wherein said capture probe further comprises a detection moiety.

13. A method of copying target nucleic acid sequences, wherein the method comprises:
(a) forming a reaction mixture comprising:
a nucleic acid sample, which may or may not comprise one or more target sequences,
(ii) one or more oligonucleotide primers, wherein the one or more oligonucleotide primers are at least partially complementary to the one or more target sequences, and
(iii) one or more Fanconi Anemia complementation group A (FANCA) proteins,
(b) incubating the reaction mixture under conditions wherein hybridization between the one or more target sequences and the one or more primers is catalyzed by the one or more FANCA proteins, and
(c) extending the at least one primer thereby copying the one or more target sequences.

14. A method of amplifying target nucleic acid sequences, wherein the method comprises:
(a) forming a reaction mixture comprising:
(i) a nucleic acid sample, which may or may not comprise one or more target sequences,
(ii) at least one pair of forward and reverse oligonucleotide primers, wherein the at least one pair of forward and reverse oligonucleotide primers is at least partially complementary to the one or more target sequences, and
(iii) one or more Fanconi Anemia complementation group A (FANCA) proteins,
(b) incubating the reaction mixture under conditions wherein hybridization between the one or more target sequences and the at least one pair of forward and reverse oligonucleotide primers is catalyzed by the one or more FANCA proteins, and
(c) extending the at least one pair of forward and reverse oligonucleotide primers in a series of cycles of primer extension, denaturation, primer hybridization and primer extension thereby amplifying the one or more target sequences.

15. A method of selectively depleting nucleic acids from a sample, the method comprising:
(a) contacting the sample with: (i) one or more oligonucleotide probes, wherein the one or more oligonucleotide probes is at least partially complementary to nucleic acids to be depleted, and (ii) one or more Fanconi Anemia complementation group A (FANCA) proteins;
(b) incubating the sample under conditions wherein hybridization between the nucleic acids to be depleted and the oligonucleotide probes is catalyzed by the one or more FANCA proteins; and
(c) removing the complexes formed in step (b) from the sample.
